(19) United States
(12) Patent Application Publication
(10) Pub. No.: US 2020/0176089 A1
(43) Pub. Date: Jun. 4, 2020

(54) Generating Automated Actions Using Artificial Intelligence and Composite Audit Trail Events

(71) Applicant: Florence Healthcare, Inc., Atlanta, GA (US)

(72) Inventors: Ryan Jones, Atlanta, GA (US); Andres Garcia, Atlanta, GA (US)

(21) Appl. No.: 16/207,579

(22) Filed: Dec. 3, 2018

(51) Int. Cl. G16H 10/20; G16H 15/00; G06Q 10/06; G16H 70/40

(52) U.S. Cl. CPC G16H 10/20; G16H 70/40; G06Q 10/063114; G16H 15/00

(57) ABSTRACT

Systems, methods, and computer-readable media are disclosed for generating automated actions using artificial intelligence and composite audit trail events. Example devices may include at least one processor configured to determine a first score for completion of a plurality of tasks digitally completed by users associated with a clinical trial site identifier, determine an average document cycle time, generate, based at least in part on the first score and the average document cycle time, an estimated startup time value, and determine a second score for compliance with digital tasks. In some instances, the at least one processor may be configured to generate a digital user interface comprising a first graphical indicator representing the first score, a second graphical indicator representing the average document cycle time, a third graphical indicator representing the estimated startup time value, and a fourth graphical indicator representing the second score, and present the digital user interface.

### BACKGROUND

Clinical trials or research studies may be performed to determine an effectiveness or safety of medical treatments, such as medical procedures, drugs, or other treatments, for humans. Clinical trials may include clinical trial sites that conduct clinical trials or research studies on various patients and collect data that may be used to determine changes in patient health. Data collected by clinical trials may be communicated to other parties, such as a sponsor of a clinical trial, and may be subject to auditing or monitoring for accuracy and/or verification. Documents created for clinical trials may be subject to certain requirements and may need to be categorized so as to regulate access and tasks by different users. Many factors may affect clinical trial results and potential approval. Accordingly, management and insight to clinical trial performance and other data may be desired.

### BRIEF DESCRIPTION OF THE DRAWINGS

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. In the drawings, the left-most digit(s) of a reference numeral may identify the drawing in which the reference numeral first appears. The use of the same reference numerals indicates similar, but not necessarily the same or identical components. However, different reference numerals may be used to identify similar components as well. Various embodiments may utilize elements or components other than those illustrated in the drawings, and some elements and/or components may not be present in various embodiments. The use of singular terminology to describe a component or element may, depending on the context, encompass a plural number of such components or elements and vice versa. For example, the term “a clinical trial identifier” can refer to one or more identifiers and clinical trials.

FIG. 1 is a schematic diagram of an example user interface with graphical indicators representing various composite audit trail events in accordance with one or more example embodiments of the disclosure.

FIG. 2 is an example process flow diagram for generating scores and automated actions using artificial intelligence in accordance with one or more example embodiments of the disclosure.

FIG. 3 is an example hybrid data and process flow for generating automated actions using artificial intelligence and composite audit trail events in accordance with one or more example embodiments of the disclosure.

FIG. 4 is an example process flow diagram for generating automated actions using artificial intelligence and composite audit trail events in accordance with one or more example embodiments of the disclosure.

FIGS. 5-6 are schematic diagrams of example user interfaces with dynamically generated graphical indicators representing various performance metrics in accordance with one or more example embodiments of the disclosure.

FIG. 7 is a schematic illustration of example computer architecture of an electronic device in accordance with one or more example embodiments of the disclosure.

### DETAILED DESCRIPTION

### Overview

This disclosure relates to, among other things, systems, methods, computer-readable media, techniques, and methodologies for generating automated actions using artificial intelligence and composite audit trail events. Clinical trials or research studies may be performed to determine or evaluate safety and/or effectiveness of medical treatments on humans. Medical treatments may include medical procedures, medicines, drugs, medical strategies, and other medical treatments. Clinical trials or research studies may be organized and conducted by several parties. For example, a clinical trial may include one or more sponsors that may support or finance the clinical trial, one or more clinical trial sites that conduct clinical trials, one or more investigators that manage clinical trials and may execute documents, and one or more monitors that may audit data collected during a clinical trial. Sponsors may be government organizations, private companies, universities, or other organizations. Monitors may be employed by sponsors or contract research organizations that implement a clinical trial on behalf of a sponsor. Clinical trial sites or research sites may be sites at which the clinical trial is conducted or performed. Clinical trial sites may perform or otherwise facilitate implementation of clinical trials and may collect data points for patients or subjects of clinical trials. The data points collected by clinical trial sites may be evaluated to determine effectiveness and/or safety of the medical treatment being tested. Data generated by clinical trial sites, which may be referred to as end points, may be subject to validation, verification, or auditing. For example, government organizations may regulate auditing or verification of clinical trial data. Accordingly, clinical trials may also include monitors or other parties that validate clinical trial data. Monitors, which may be principal investigators, may monitor or review data generated by a clinical trial site for accuracy, completeness, and other metrics, and may authenticate results or findings of a clinical trial.

In order to monitor clinical trial site data, documents with patient information and/or data points collected by a clinical trial site may be communicated to monitors or other parties. In addition, verification, validation, and/or auditing of documents and other records associated with a clinical trial may be needed. Management of such data and documents may be time consuming, and real-time insight as to whether or not certain actions have been completed by one or more parties may be unavailable in instances where documents and other data may be shared via physical methods, such as fax or mail. Moreover, certain clinical trial sites may have different operational efficiencies that result in different clinical trial startup times, different levels of compliance, and may overall impact submission of clinical trial results for government review, in one example. To improve levels of compliance, operational efficiencies, and other factors that may impact submission and/or positive outcomes of clinical trials, automated action generation using artificial intelligence may be used.

The systems, methods, computer-readable media, techniques, and methodologies for generating automated actions using artificial intelligence and composite audit trail events described herein may provide efficient document and/or information transfer between parties to a clinical trial, generate real-time compliance data, automatically monitor the occurrence of certain digital events and track states across various tasks, control permissions and access to various documents and data, and/or automatically generate tasks and/or assignments using task or document state data. Some embodiments may facilitate tracking of requests for clinical trial information, while protecting personally identifiable information and other sensitive patient information, or prescribing specific actions by parties. Embodiments of the disclosure may further facilitate compliance with governmental regulations, including, for example, regulations related to document archiving, patient information and privacy protection, wet and dry document signature regulations, and other government regulations.

Embodiments of the disclosure include a number of technical features that may be implemented to accelerate clinical trials for sites, sponsors, contact research organizations, and so forth. Example technical features include digital document management, electronic task generation and assignment, mandated workflows, dynamic permissions and access control, device authentication features, and user interface generation, amongst other technical features. Certain embodiments allow for the reduction or elimination of mailing, emailing, and/or faxing documents to parties by providing a study file structure that is accessible via electronic devices and one or more user interfaces. Some embodiments may allow file structures to be published directly to electronic platforms that can subsequently be securely shared while maintaining compliance. The above examples of technical features and/or technical effects of example embodiments of the disclosure are merely illustrative and not exhaustive.

Clinical trials or studies may include a coordinator that may be located at a clinical trial site or a research site and may facilitate or conduct a clinical trial. The coordinator may collect data from patients that are subjects of the clinical trial and may create, handle, or store documents for clinical trials. The coordinator may generate relevant documents (e.g., source and regulatory documents) by inputting data at a coordinator device. The monitor may be a sponsor of a clinical trial or affiliated with a sponsor and may verify documents that are generated by clinical trial site. For example, the monitor may receive relevant documents at a monitor device from the coordinator device via one or more network(s). The monitor may be a contract research organization (CRO) and may verify the end points provided by the coordinator. A principal investigator may validate documents. For example, the principal investigator may receive relevant documents at a principal investigator device via the network from the coordinator via the coordinator device and/or the monitor via the monitor device. The principal investigator may sign off documents associated with a clinical trial, such as source documents with end points or results of the clinical trial. Example types of documents or source documents may include, without limitation, electronic health records, end points in Case Report Forms, patient data, or the like.

In some instances, a remote server may be configured to generate research binders for clinical trials. Research binders may be virtual folders generated by the remote server that may include some or all relevant documentation, messages, evidence, data, and other information for particular clinical trials. Research binders generated by the remote server may be accessed by appropriate parties, as described herein, and may provide statuses of various tasks or queries/requests associated with a clinical trial. The remote server may further be configured to facilitate validation of source documents that are generated by the coordinator, for example, by tracking a status of a task or query/request made by the principal investigator 130, among other functions. The remote server may be in communication with one or more datastores. For example, the remote server may be in communication with a Clinical Trial #1 datastore that may be configured to receive and/or store data associated with a particular clinical trial. Metrics associated with a particular clinical trial, such as timeliness of task response, accuracy, responsiveness, and other metrics may be tracked in a dashboard presented by the remote server. Metrics may be generated by the remote server and may be used to evaluate clinical trial or research site overall quality. In some embodiments, various metrics may be used to generate engagement scores, compliance scores (e.g., Good Clinical Practice (GCP) compliance scores, etc.), productivity scores, responsiveness scores, and/or other scores.

Engagement scores may represent a level of engagement of a user and/or a clinical trial site with a particular study. Metrics that may be used to determine engagement scores may include a number of users added by the clinical trial site to the study over a particular time interval, total audit trail activities for the clinical trial site under the study, and/or other metrics.

Compliance scores may represent compliance with one or more guidelines. Metrics that may be used to determine compliance scores may include a total number of documents with past expiration dates (e.g., past due documents and forms, etc.), a number of days by which the respective documents are expired or past due, and/or other metrics.

Productivity scores may represent user and/or clinical trial site productivity with respect to one or more clinical trial studies. Metrics that may be used to determine productivity scores may include a total number of document versions, a total number of documents that have been executed, a total number of forms ready for approval, and/or other metrics.

Responsiveness scores may represent user and/or clinical trial site responsiveness to communications for one or more clinical trial studies (e.g., individual scores for users/sites or particular studies, aggregate scores for users/sites or particular studies, etc.). Metrics that may be used to determine responsiveness scores may include a total number of placeholders that have been filled, a total number of placeholders with past due dates, a total number of days remaining to fill placeholders (or a total number of days it took to fill placeholders), a total number of documents with pending/outstanding signatures, a total number of days from sending signature requests to 100% signed (e.g., completely executed) documents, a total number of day from a forms ready for approval state to an approved state, a total number of tasks outstanding and/or completed, a total number of documents (e.g., documents and forms), a total number of documents with expiration date (e.g., documents and forms), a total number of document downloads (e.g., documents and forms), a total number of placeholders created, a total number of placeholders with due dates, a total number of documents with signature requests, a total number of documents with declined signatures, a total number of forms, a total number of forms approved, a total number of forms rejected, a total number of forms canceled, and/or other metrics.

Although certain metrics are described with respect to the example engagement scores, compliance scores, productivity scores, and responsiveness scores, one or more of the metrics, as well as other metrics, may be used to determine more than one score, and some of the scores may use metrics that are described with respect to other scores. For example, an engagement score may be determined using the responsiveness metric of a total number of forms approved.

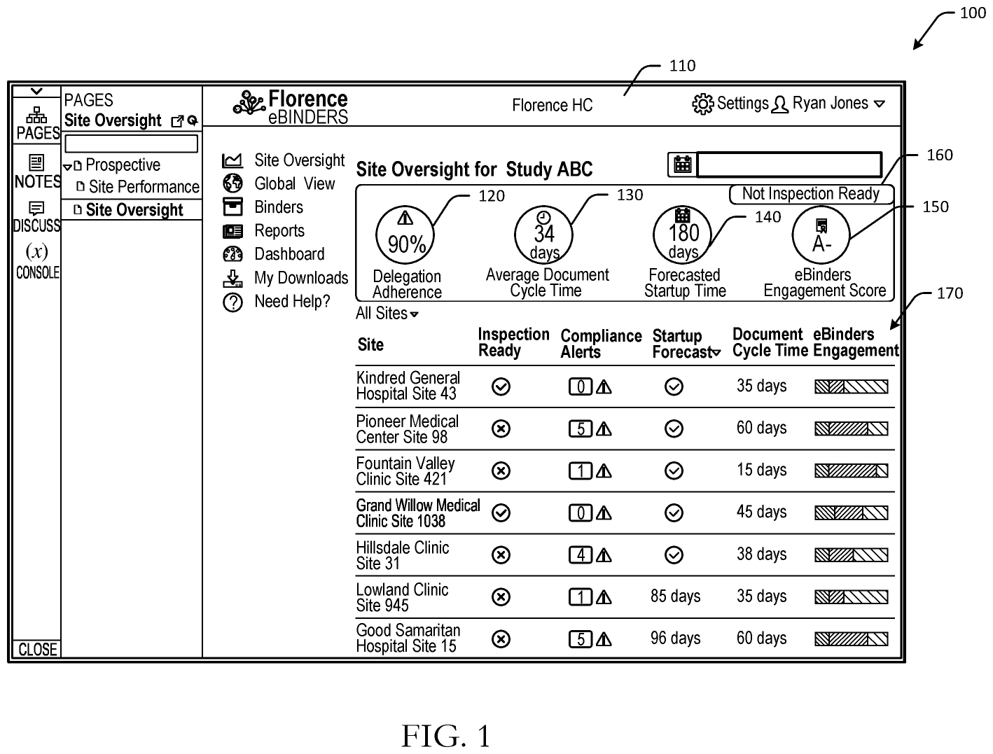

Referring to FIG. 1, an example user interface 100 with graphical indicators representing various composite audit trail events is illustrated in accordance with one or more example embodiments of the disclosure. The illustrated user interface may include one or more dynamically generated graphical indicators representing various data and may be reformatted in real-time, in some instances, based at least in part on display and/or device configurations. The user interface 100 provides at least the technical benefit of reduced latency and bandwidth consumption in network communication as a result of, in part, aggregation of data from multiple clinical trial sites in a single user interface. For example, as opposed to the typical distribution of study documents, which would be manually completed on an individual clinical trial site basis (and repeated for each clinical trial site involved), certain embodiments may use the user interface to provide access to documents to each involved clinical trial site without having to send or resend the documents individually. In addition, a number of user actions (e.g., clicks, inputs, etc.) needed to access data for each clinical trial site may be reduced as a result of the aggregated data presentation illustrated in FIG. 1.

The user interface 100 may include a dashboard or overview of various information for individual clinical trials, amongst other information. For example, the user interface 100 may include a site oversight dashboard 110 for a certain clinical trial study, such as Study ABC. The sight oversight dashboard 100 may include graphical representations or indicators of various data for individual clinical trial sites, individual clinical trials, individual personnel members, and/or other data.

In the example of FIG. 1, the site oversight dashboard 110 may include a first graphical indicator 120 representing delegation adherence, a second graphical indicator 130 representing average document cycle time, a third graphical indicator 140 representing forecasted startup time, a fourth graphical indicator 150 representing an engagement score, and a fifth graphical indicator 160 representing an inspection readiness determination for the clinical trial. Other embodiments may include additional, fewer, or different graphical indicators.

The respective graphical indicators may represent aggregated data for a clinical trial across any number of, such as a plurality of, clinical trial sites. For example, as illustrated in FIG. 1, individual sites may have site-specific data 170, a portion of which may be aggregated and used to generate one or more of the graphical indicators. For example, a list of sites is depicted in FIG. 1. The individual sites may be associated with graphical indicators representing whether or not the specific site is ready for inspection. Inspection readiness may be automatically determined using one or more algorithms and may be based at least in part on site performance and task completion data. If a site is determined to be inspection ready, the site may have completed certain tasks and/or may have completed other objectives. For example, Kindred General Hospital Site 43 may be inspection ready, as indicated by the checkmark graphical indicator, while Pioneer Medical Center Site 98 may be determined to not be inspection ready, as illustrated by the “x” graphical indicator. One or more of the individual sites may be associated with a number of compliance alerts, which may provide information related to any compliance issues identified at the specific site. As a result, a monitor or other user reviewing the dashboard 110 may determine at a glance the status and/or performance of an individual site, as well as status and/or performance of a clinical trial.

The site specific data 170 of the dashboard 110 may include graphical indicators representing whether or not a clinical trial has been started at a specific site, as illustrated in a “startup forecast” column. If the clinical trial has been started, a checkmark graphical indicator may be generated, and if not, an estimated length of time before the trial is started may be presented. The estimated startup time, or the forecasted startup time, may be determined based at least in part on digital actions or tasks that have been completed by the respective site, as well as historical performance data associated with the specific site. For example, the Lowland Clinic Site 945 may have an estimated startup time of 85 days. Using this information, a monitor may better understand a timeline of the clinical trial. Individual document cycle time data may also be determined for individual clinical trial sites. The document cycle times may represent an average length of time needed by the site to cycle documents. For example, document cycle times may range from 15 days to 60 days in the illustrated examples, and may impact the overall performance of the site and/or the trial itself. The document cycle time may be automatically generated or determining digital user actions using device data and/or feedback. For example, document signature or execution may be detected digitally and may be used to determine a length of time between assignment of a task associated with the signature and the execution of the document, which may represent the cycle time for that particular document. An eBinders engagement graphical indicator may also be generated for the individual sites and may represent an amount of engagement with digital tasks and/or documents or other data that users associated with the site have had with a certain digital platform.

Some or all of the individual site specific data 170 may be used to generate one or more of the graphical indicators 120, 130, 140, 150, 160. For example, the first graphical indicator 120 may represent delegation adherence for the clinical trial as a whole, and may use aggregated data from one or more of the sites. The delegation adherence value represented by the first graphical indicator 120 may illustrate a level of adherence for tasks that have been delegated to sites and that have been completed or otherwise adhered to. The delegation adherence value may be aggregated across all sites associated with a particular trial. The second graphical indicator 130 may represent average document cycle time for the clinical trial, and may be aggregated across all sites associated with a trial (with respect to document cycle time for the particular trial at the site). The second graphical indicator 130 may therefore represent mindshare a study is receiving at a particular site. The third graphical indicator 140 may represent forecasted startup time for the trial and may be aggregated across the participating sites. The fourth graphical indicator 150 may represent an overall engagement score with digital tasks aggregated across the sites participating in the trial. The fifth graphical indicator 160 may represent an inspection readiness determination for the clinical trial and may be determined by aggregating data across the participating sites.

Accordingly, the user interface 100 may include one or more graphical indicators that may be generated in real-time using data from a plurality of devices. The site oversight dashboard 110 may allow monitors and other users to quickly determine a status and/or performance of a clinical trial and/or participating sites, and may provide aggregated operational data that may otherwise be unable to be determined manually.

The user interface 100 therefore provides a user with the ability to monitor clinical trial site progress, and provides real-time insights into individual site, and study-wide, progress and source documents. In some embodiments, the system may automatically identify potential delays and compliance risk across study sites. The user interface 100 may be used to determine where sites stand with their startup and study progress in real-time. Historical site operational performance may be used to forecast site performance and/or study startup time.

As a result, certain embodiments of the disclosure may accelerate study startup time by about 25% or more (e.g., by publishing study documents to sites digitally in one click, providing customized workflows, etc.), reduce or eliminate compliance mistakes (e.g., by generating a full audit trail of every document electronically, etc.), reduce document cycle time by about 40% or more (e.g., by automatically generating and assigning tasks using artificial intelligence, etc.), and so forth.

One or more illustrative embodiments of the disclosure have been described above. The above-described embodiments are merely illustrative of the scope of this disclosure and are not intended to be limiting in any way. Accordingly, variations, modifications, and equivalents of embodiments disclosed herein are also within the scope of this disclosure. The above-described embodiments and additional and/or alternative embodiments of the disclosure will be described in detail hereinafter through reference to the accompanying drawings.

### Illustrative Processes and Use Cases

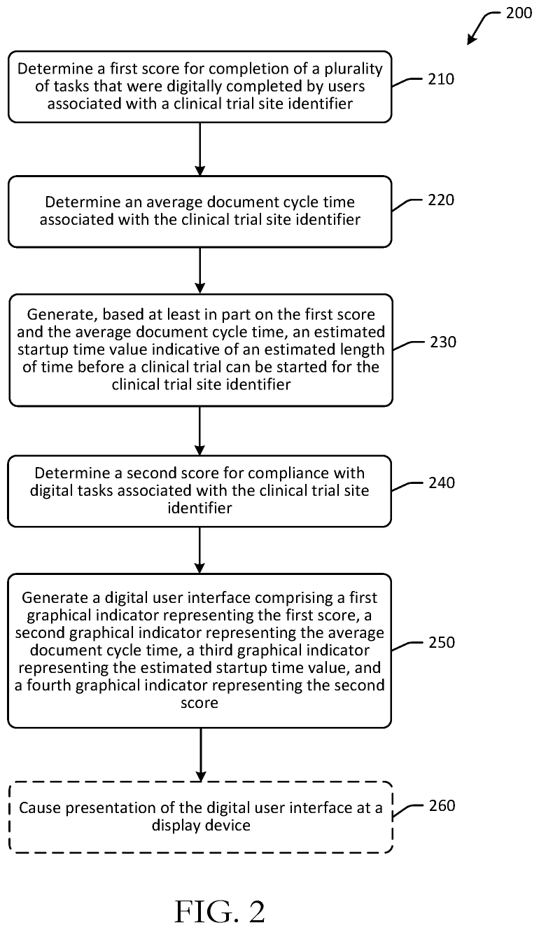

FIG. 2 is an example process flow diagram for generating scores and automated actions using artificial intelligence in accordance with one or more example embodiments of the disclosure. One or more operations or communications illustrated in FIG. 2 may occur concurrently or partially concurrently, while illustrated as discrete communications or operations for ease of illustration. One or more blocks of FIG. 2 may be optional and may be performed by a single computer system or across a distributed computing system.

At block 210 of the process flow 200, computer-executable instructions stored on a memory of a device, such as a remote server or a user device, may be executed to determine a first score for completion of a plurality of tasks that were digitally completed by users associated with a clinical trial site identifier. For example, a remote server may receive (e.g., from another computer system or service provider, etc.) indications of user interactions at certain devices (e.g., pixel value changes, interactions with documents, etc.) that may be used to correlate and/or determine that certain tasks have been completed. For example, a set of users may be associated with a clinical trial site identifier (e.g., the name of a clinical trial site, etc.). The users may be personnel or other entities associated with the clinical trial site identifier. One or more of the users may be assigned tasks either automatically or manually. For example, a site coordinator may be assigned a task of executing a document, verifying certain data, and so forth. The assigned tasks may be tracked to determine whether or not the task has been completed. To determine completion, some embodiments may actively or passively track device interactions. For example, a device that is associated with the site coordinator may be used to track the actions the site coordinator takes using the device. Accordingly, if the coordinator signs a document to complete a task, the coordinator may not have to actively mark the task complete. Instead, a remote server may receive interaction data from the device and may automatically determine that the task is complete based on the device interaction data. This may increase efficiency and improve insight into trial status. In some embodiments, multiple tasks may be outstanding at a time, and as a result, multiple user identifiers and/or device identifiers may be tracked. Interaction data may be correlated across existing tasks to determine completion.

Based at least in part on data associated with completion of tasks, such as a time to completion, whether the task was correctly completed, etc., a first score may be determined. The first score may indicate a performance of the particular trial site for the particular trial. For example, sites that have relatively long times before completion of tasks, or take longer to complete tasks, may result in a lower score than sites that have relatively shorter times before completion of tasks. Whether or not a task is correctly completed may impact the first score as well. The first score may be for a particular clinical trial or for more than one clinical trial associated with the site.

In an example embodiment, the first score may correspond to a delegation adherence value. Accordingly, the remote server may determine an average time to completion for the plurality of tasks, determine a number of uncompleted tasks associated with the clinical trial site identifier, and determine a number of tasks completed after respective deadlines (e.g., tasks completed late, etc.). The first score may therefore be determined based at least in part on the average time, the number of uncompleted tasks, and the number of tasks completed after the respective deadlines.

At block 220 of the process flow 200, computer-executable instructions stored on a memory of a device, such as a remote server or a user device, may be executed to determine an average document cycle time associated with the clinical trial site identifier. For example, a remote server may determine an average document cycle time that is associated with documents assigned to a particular clinical trial site. The average document cycle time may be for a particular clinical trial or for more than one clinical trial associated with the site.

At block 230 of the process flow 200, computer-executable instructions stored on a memory of a device, such as a remote server or a user device, may be executed to generate, based at least in part on the first score and the average document cycle time, an estimated startup time value indicative of an estimated length of time before a clinical trial can be started for the clinical trial site identifier. For example, a remote server may determine the first score and the average document cycle time, and may generate an estimated startup time value for the site. The estimated startup time value or forecasted startup time may be an estimate of how long it may be before a site can start a clinical trial.

At block 240 of the process flow 200, computer-executable instructions stored on a memory of a device, such as a remote server or a user device, may be executed to determine a second score for compliance with digital tasks associated with the clinical trial site identifier. For example, a remote server may determine a second score for compliance with digital tasks associated with the clinical trial site identifier. The second score may be determined based at least in part on automatic detection of completion of digital tasks that are associated with the clinical trial site identifier. Compliance may be indicative of satisfaction of certain requirements before or during a study. For example, doctors may need to provide resumes before a study begins, and whether or not all participating doctors have provided resumes may impact compliance, and therefore, may impact the second score. In some embodiments, scores may be generated for (i) clinical trials themselves, (ii) clinical trial sites for specific or multiple clinical trials, and/or (iii) individual personnel at clinical trial sites, which may be used to determine performance of a site as a whole. For example, scores for personnel may include digital logs of actions the user performed, how long it took them, the effort or volume of actions completed, and/or other events. Personnel scores may be used to increase or decrease permissions or access controls associated with the user. For example, a user with a high score may have greater permissions than a user with a low score.

At block 250 of the process flow 200, computer-executable instructions stored on a memory of a device, such as a remote server or a user device, may be executed to generate a digital user interface comprising a first graphical indicator representing the first score, a second graphical indicator representing the average document cycle time, a third graphical indicator representing the estimated startup time value, and a fourth graphical indicator representing the second score. For example, a remote server may determine data used to generate the respective first graphical indicator, the second graphical indicator, the third graphical indicator, and the fourth graphical indicator from one or more devices or datastores. Based at least in part on the data, the remote server may generate a digital user interface, such as that illustrated in FIG. 1, that includes a first graphical indicator representing the first score, a second graphical indicator representing the average document cycle time, a third graphical indicator representing the estimated startup time value, and a fourth graphical indicator representing the second score. The user may use this user interface to capture various information in a single view that may otherwise not be attainable in an analog or manual manner.

At optional block 260 of the process flow 200, computer-executable instructions stored on a memory of a device, such as a remote server or a user device, may be executed to cause presentation of the digital user interface at a display device. For example, a remote server may send the generated user interface to a display device for presentation. The particular format and/or configuration of the user interface may be dynamically modified by the remote server based at least in part on a device type and/or device settings/configuration of the display device.

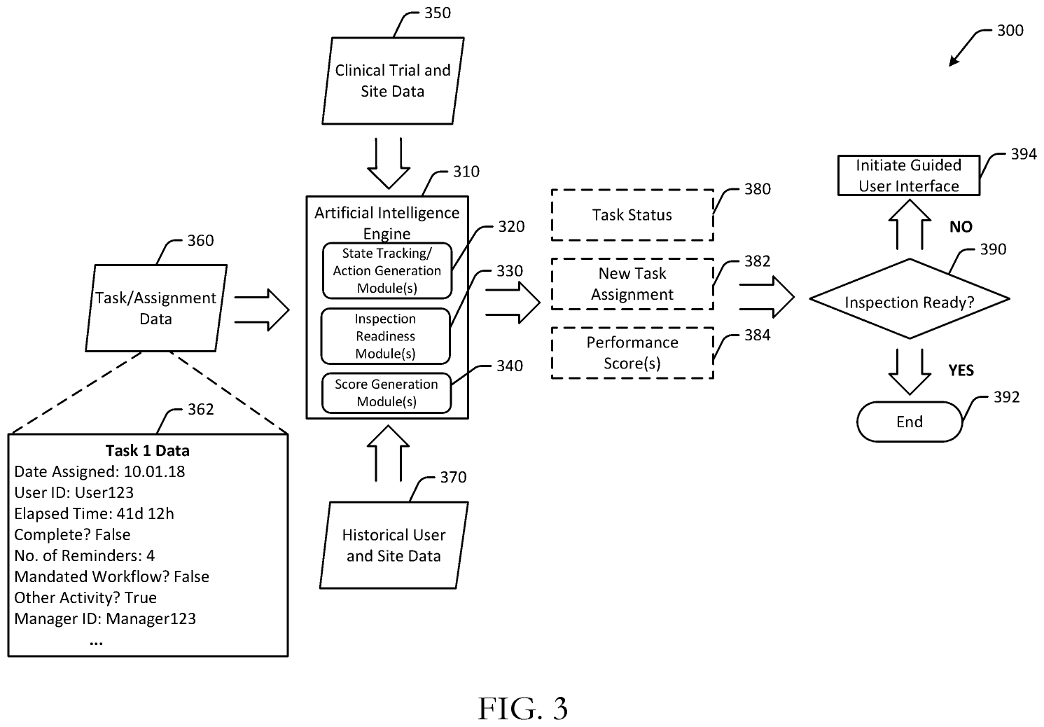

FIG. 3 is an example hybrid data and process flow for generating automated actions using artificial intelligence and composite audit trail events in accordance with one or more example embodiments of the disclosure. Different embodiments may include different, additional, or fewer inputs or outputs than those illustrated in the example of FIG. 3.

In FIG. 3, an example hybrid data and process flow 300 is schematically depicted. A artificial intelligence engine 310 may be configured to determine task status and automatically generate tasks. The artificial intelligence engine 310 may be stored at and/or executed by one or more remote servers. The artificial intelligence engine 310 may include one or more modules and/or algorithms, and may be configured to determine various performance metrics for clinical trials, sites, and/or individuals.

For example, the artificial intelligence engine 310 may include one or more state tracking/action generation modules 320, one or more inspection readiness modules 330, and/or one or more score generation modules 340. Additional or fewer, or different, modules may be included. The state tracking/action generation modules 320 may be configured to determine a state of a task and to automatically determine completion of tasks. In some embodiments, the state tracking/action generation modules 320 may automatically determine new tasks to assign based at least in part on completion of tasks using artificial intelligence. For example, when a document execution task is completed, the state tracking/action generation modules 320 may route the document to a supervisor for review if the executor has historically failed to execute documents properly, or may submit the document for submission if the executor has historically completed documents properly.

The inspection readiness modules 330 may be configured to determine, in real-time, inspection readiness levels of sites and/or clinical trials. The inspection readiness modules 330 may aggregate data associated with clinical trials and sites over time to increase accuracy of inspection readiness determinations.

The score generation modules 340 may be configured to generate one or more scores indicative of various performance metrics, such as clinical trial performance, clinical trial site performance, individual personnel performance, and/or other metrics.

The artificial intelligence engine 310 may receive one or more inputs that may be used to determine a number of outputs. Outputs may include, in the example of FIG. 3, a task status 380, new task assignment 382, and/or performance scores 384. Example inputs to the artificial intelligence engine 310 may include one or more of clinical trial and site data 350 that may be from third parties and may include trial identifiers, assigned tasks and statuses, and so forth, task/assignment data 360 that may include completed and/or outstanding tasks and associated personnel or entities, and/or historical user and site data 370 that may historical data associated with users, sites, and/or clinical trials. Example task/assignment data 360 may include a task record 362 that includes various data records associated with a task, such as a date of assignment, a user identifier, elapsed time, whether or not the task is complete, a number of reminders generated, whether or not the task has been pushed to a mandated workflow (e.g., forcing the user to complete the task before completing a different task by preventing or limiting user permissions or access, etc.), whether the user has been active on other trials or activities, a manager identifier, and/or other information. Such data may be used to generate various scores and/or graphical indicators associated with the user, site, and/or trial. Mandated workflows may include determining that a deadline to complete a first task has elapsed. The remote server may receive an indication that the first user identifier has requested access to a second document, and may prevent access to the second document by the first user identifier until the first task is completed. Access to documents may be controlled by user permissions associated with user accounts. In order to access documents, in one example, embodiments of the disclosure may receive an access request to access a document, identify a user account associated with the access request, and determine that the user account is authorized to access the document or perform other tasks within the system.

The artificial intelligence engine 310 may process the clinical trial and site data 350, the task/assignment data 360, and/or the historical user and site data 370 to determine one or more of the task status 380, new task assignment 382, and/or performance scores 384. For example, task status 380 may change automatically once the artificial intelligence engine 310 determines that the task has been completed (e.g., via the task/assignment data 360). If a task status indicates that the task is not completed, the artificial intelligence engine 310 may initiate one or more actions automatically, such as notifying the user with a reminder, notifying a manager, modifying the user permissions, and so forth. The artificial intelligence engine 310 may assign new tasks 382 based at least in part on completion of previously assigned tasks, changes in task states, chronological factors, and/or other factors. The artificial intelligence engine 310 may generate one or more performance scores using the data inputs.

To determine user-specific scores, a remote server may determine a length of time between a time of assignment of the first task to the first user and a time of completion of the first task, determine a number of late tasks associated with the first user identifier, determine a number of completed tasks associated with the first user identifier, and generate a performance score for the first user identifier based at least in part on the length of time, the number of late tasks, and the number of completed tasks. User-specific scores may be used to determine permissions and/or levels of access for users. For example, a level of access associated with the first user identifier may be based at least in part on a performance score for a user.

Performance scores may include scores for trials and may at least partially represent completion of milestones associated with the clinical trial identifier.

Using one or more algorithms or modules, the artificial intelligence engine 310 may optionally determine, at determination block 390, whether or not the clinical trial and/or site is inspection ready. For example, based at least in part on task statuses 380, newly assigned tasks 382, and/or performance scores 384, the artificial intelligence engine 310 may determine whether the site or trial is ready for inspection. If so, the process flow 300 may end at block 392. If not, the process flow 300 may proceed to block 394, at which a guided user interface may be initiated to walk users through the errors detected so that readiness is achieved. If a site or trial is determined not to be inspection ready, the remote server may implement remedial actions. For example, the remote server may receive a request to submit clinical trial data, the remote server may determine that an error is present in the clinical trial data, and may prevent submission of the clinical trial data until the error is resolved. The remote server may generate a series of guided user interfaces to correct the error.

In an example embodiment, to determine whether a site or trial is inspection ready, the artificial intelligence engine 310 may determine an inspection score for the clinical trial site identifier or the trial based at least in part on the first score and the second score (as discussed with respect to FIG. 2), and may determine that the inspection score is less than an inspection ready threshold or greater than or equal to the inspection ready threshold. A graphical indicator may be presented at a digital user interface indicating that the clinical trial site identifier is not ready for inspection, or is ready for inspection.

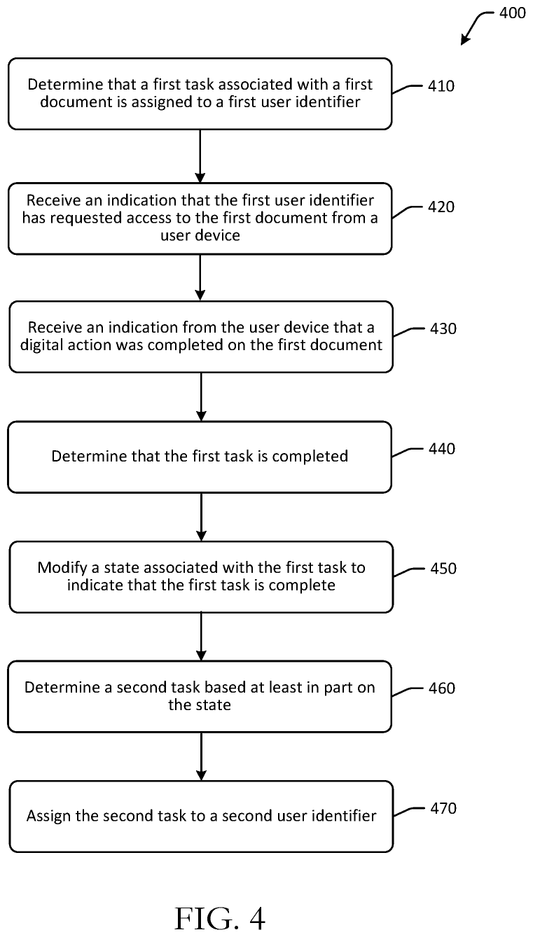

FIG. 4 is an example process flow 400 for generating automated actions using artificial intelligence and composite audit trail events in accordance with one or more example embodiments of the disclosure. One or more operations or communications illustrated in FIG. 4 may occur concurrently or partially concurrently, while illustrated as discrete communications or operations for ease of illustration. One or more blocks of FIG. 4 may be optional and may be performed by a single computer system or across a distributed computing system.

At block 410 of the process flow 400, computer-executable instructions stored on a memory of a device, such as a remote server or a user device, may be executed to determine that a first task associated with a first document is assigned to a first user identifier. For example, a remote server may determine that a first task associated with a first document is assigned to a first user identifier. The first task may have a state or status of uncompleted, completed, in progress, late, or another status.

At block 420 of the process flow 400, computer-executable instructions stored on a memory of a device, such as a remote server or a user device, may be executed to receive an indication that the first user identifier has requested access to the first document from a user device. For example, a remote server may receive an indication from a user device associated with the first user identifier indicating that the first user has requested access to the first document. The remote server may therefore automatically determine that the first task is in progress, and may modify the state of the first task accordingly. In some embodiments, access to the first document may be allowed only to certain users and/or by certain user devices.

At block 430 of the process flow 400, computer-executable instructions stored on a memory of a device, such as a remote server or a user device, may be executed to receive an indication from the user device that a digital action was completed on the first document. For example, a remote server may receive an indication that the user completed a digital task using the user device. The user may not actively indicate the digital task in some instances.

At block 440 of the process flow 400, computer-executable instructions stored on a memory of a device, such as a remote server or a user device, may be executed to determine that the first task is completed. For example, a remote server may determine that the digital task corresponds to an expected task, or the first task, and may therefore determine that the first task is completed. For example, if the first task is execution of a document, the remote server may determine that the user drew a line representing a signature on a display of the user device, and may determine that the user signed the document. This may be opposed to an instance where the user scrolled through the document, which would be a different digital action, but may not correspond to the first task. As a result, the first task would not be completed in such an instance.

At block 450 of the process flow 400, computer-executable instructions stored on a memory of a device, such as a remote server or a user device, may be executed to modify a state associated with the first task to indicate that the first task is complete. For example, a remote server may determine that the first task is complete, and as a result, may automatically modify a state associated with the first task to indicate that the first task is complete.

At block 460 of the process flow 400, computer-executable instructions stored on a memory of a device, such as a remote server or a user device, may be executed to determine a second task based at least in part on the state. For example, a remote server may determine a second task based at least in part on the state. In the example of a state indicating that a task is complete, a subsequent task may be automatically generated. In some instances, artificial intelligence may be used to determine subsequent tasks. For example, determining whether a task is to be manually reviewed may be determined using artificial intelligence.

At block 470 of the process flow 400, computer-executable instructions stored on a memory of a device, such as a remote server or a user device, may be executed to assign the second task to a second user identifier. For example, a remote server may determine a second user identifier to assign the second task to. The second user identifier may be automatically determined based at least in part on the task and/or performance scores associated with the first user and/or the second user.

Examples of tasks may vary depending on requesting entities. For example, research sites or clinical trial sites may authorize monitors to generate a task request, which may be placed in a queue that includes all task requests for a clinical trial. Some task requests may have a specific lifecycle. Task statuses and related status identifiers may include in progress, indicating that the task is in the process of being addressed, complete, indicating that the task has been completed by a coordinator and a monitor may validate the documents provided by the coordinator, rejected, indicating that a monitor has rejected a task response as incomplete or incorrect, abandoned, closed, or other task statuses.

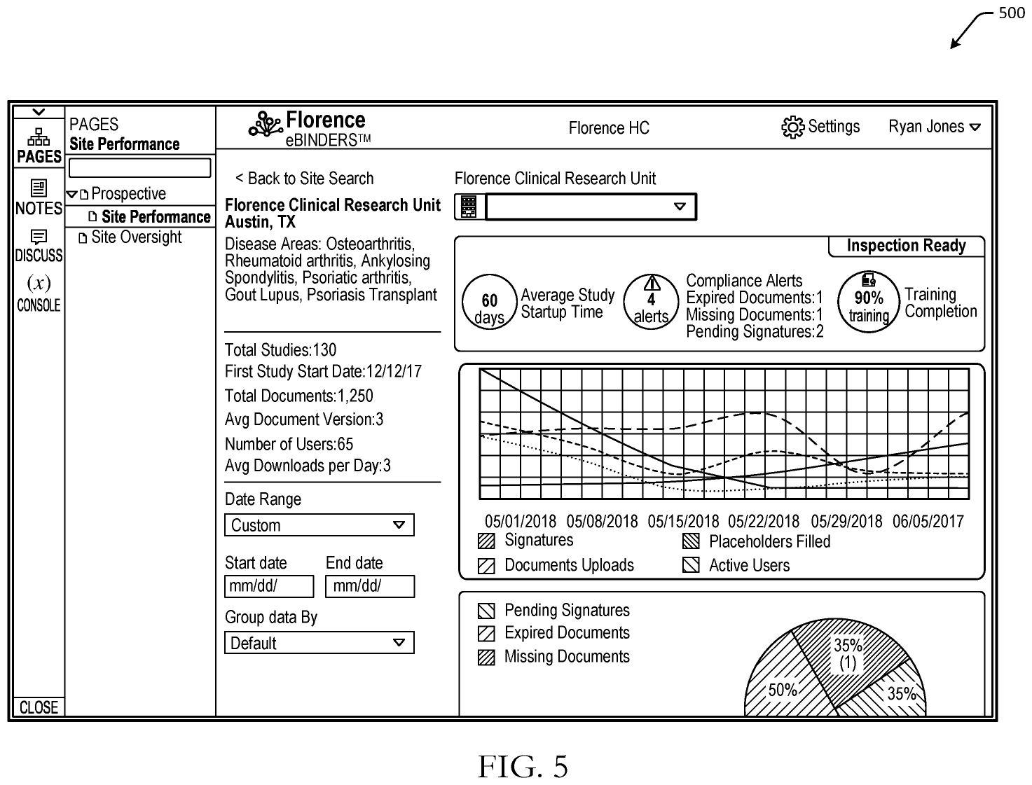

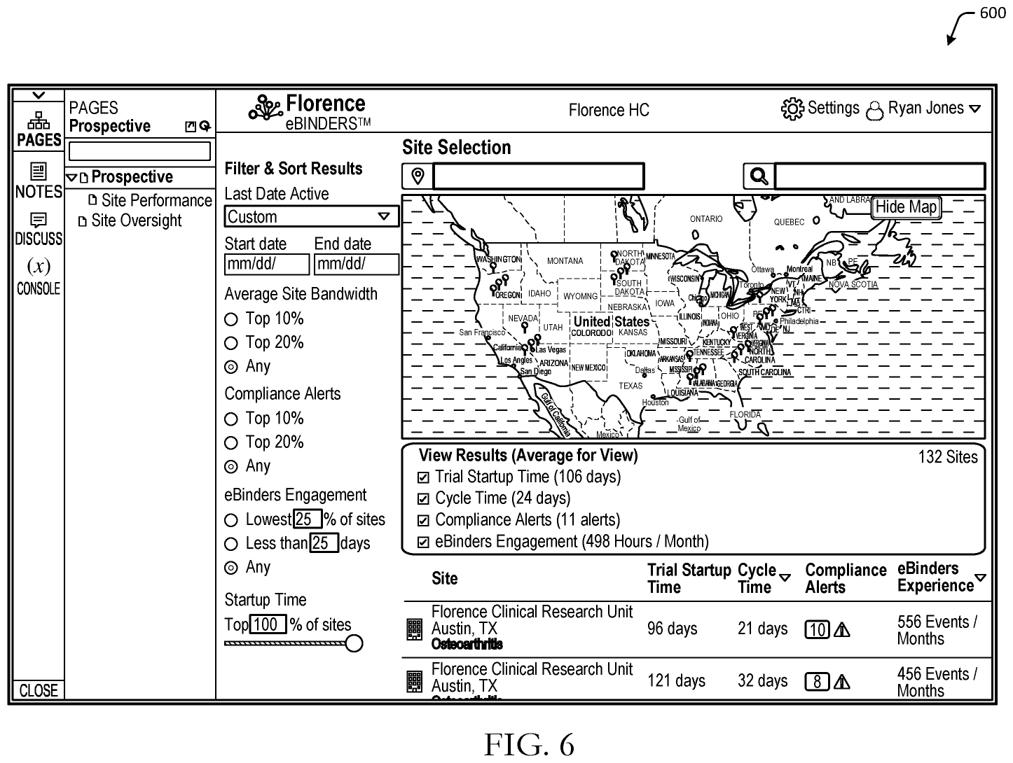

FIGS. 5-6 are schematic diagrams of example user interfaces with dynamically generated graphical indicators representing various performance metrics in accordance with one or more example embodiments of the disclosure.

In FIG. 5, an example user interface 500 illustrates specific site performance in a single user interface. The user interface 500 may include disease area information, a number of studies completed by the site, a first study start date, a total number of documents associated with the site, an average number of revisions per document for the site, a number of users associated with the site, an average number of downloads, and/or other data.

Graphical indicators representing the site's average startup time, compliance alerts, and training completion may be generated and presented. A graph of actions completed by the site over time may be generated using aggregated data and may provide insight as to the frequency of action completion by a site (e.g., whether all actions are being completed at the same time before a deadline or more consistently during a trial, etc.). Data included in the graph may include signatures that occur, documents that are uploaded, placeholders filled (e.g., placeholder resumes replaced with actual resumes, etc.), active user data, and so forth. A chart illustrating pending signatures, expired documents, and/or missing documents may be generated and presented in various formats, such as the pie chart illustrated in FIG. 5. The digital user interface 500 may include graphical indicators representing inspection readiness data for a single site or for a plurality of clinical trial site identifiers.

In some embodiments, automated monitoring logs may be generated. For example, a remote server may determine that a pharmaceutical monitor user account accessed the digital user interface 500, and may store a data record representing the access. The remote server may automatically generate a monitoring report based at least in part on the data record.

FIG. 6 illustrates an example user interface 600 illustrating site selection in a geographical context. The user interface 600 may allow a user to identify sites that have certain amounts of bandwidth, certain amounts of compliance alerts or performance, certain amounts of engagement, certain startup time lengths, and so forth. Users may therefore locate sites meeting certain criteria using the user interface 600 and the user interface 600 may dynamically generate graphical interfaces and indicators to represent data in a number of different combinations based at least in part on real-time data generation.

One or more operations of the process flows or use cases of FIGS. 1-6 may have been described above as being performed by a user device, or more specifically, by one or more program modules, applications, or the like executing on a device. It should be appreciated, however, that any of the operations of process flows or use cases of FIGS. 1-6 may be performed, at least in part, in a distributed manner by one or more other devices, or more specifically, by one or more program modules, applications, or the like executing on such devices. In addition, it should be appreciated that processing performed in response to execution of computer-executable instructions provided as part of an application, program module, or the like may be interchangeably described herein as being performed by the application or the program module itself or by a device on which the application, program module, or the like is executing. While the operations of the process flows or use cases of FIGS. 1-6 may be described in the context of the illustrative remote server, it should be appreciated that such operations may be implemented in connection with numerous other device configurations.

The operations described and depicted in the illustrative process flows or use cases of FIGS. 1-6 may be carried out or performed in any suitable order as desired in various example embodiments of the disclosure. Additionally, in certain example embodiments, at least a portion of the operations may be carried out in parallel. Furthermore, in certain example embodiments, less, more, or different operations than those depicted in FIGS. 1-6 may be performed.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

### Illustrative Device Architecture

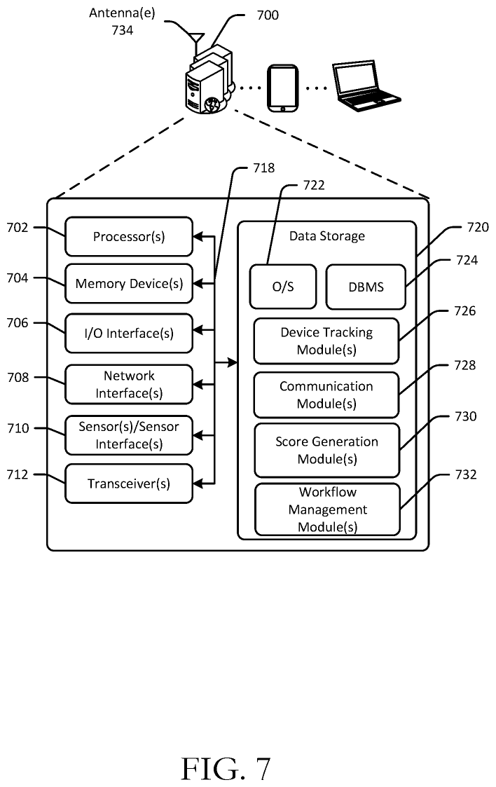

FIG. 7 is a schematic illustration of example computer architecture of an illustrative remote server 700 in accordance with one or more example embodiments of the disclosure. The remote server 700 may include any suitable computing device capable of receiving and/or generating data including, but not limited to, a mobile device such as a smartphone, tablet, e-reader, wearable device, or the like; a desktop computer; a laptop computer; a server; or the like. The remote server 700 may correspond to an illustrative device configuration for the devices of FIGS. 1-6.

The remote server 700 may be configured to communicate via one or more networks with one or more servers, user devices, or the like. In some embodiments, a single remote server or single group of remote servers may be configured to perform more than one type of artificial intelligence functionality.

Example network(s) may include, but are not limited to, any one or more different types of communications networks such as, for example, cable networks, public networks (e.g., the Internet), private networks (e.g., frame-relay networks), wireless networks, cellular networks, telephone networks (e.g., a public switched telephone network), or any other suitable private or public packet-switched or circuit-switched networks. Further, such network(s) may have any suitable communication range associated therewith and may include, for example, global networks (e.g., the Internet), metropolitan area networks (MANs), wide area networks (WANs), local area networks (LANs), or personal area networks (PANs). In addition, such network(s) may include communication links and associated networking devices (e.g., link-layer switches, routers, etc.) for transmitting network traffic over any suitable type of medium including, but not limited to, coaxial cable, twisted-pair wire (e.g., twisted-pair copper wire), optical fiber, a hybrid fiber-coaxial (HFC) medium, a microwave medium, a radio frequency communication medium, a satellite communication medium, or any combination thereof.

In an illustrative configuration, the remote server 700 may include one or more processors (processor(s)) 702, one or more memory devices 704 (generically referred to herein as memory 704), one or more input/output (I/O) interface(s) 706, one or more network interface(s) 708, one or more sensors or sensor interface(s) 710, one or more transceivers 712, and data storage 720. The remote server 700 may further include one or more buses 718 that functionally couple various components of the remote server 700. The remote server 700 may further include one or more antenna(e) 734 that may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals, a Near Field Communication (NFC) antenna for transmitting or receiving NFC signals, and so forth. These various components will be described in more detail hereinafter.

The bus(es) 718 may include at least one of a system bus, a memory bus, an address bus, or a message bus, and may permit exchange of information (e.g., data (including computer-executable code), signaling, etc.) between various components of the remote server 700. The bus(es) 718 may include, without limitation, a memory bus or a memory controller, a peripheral bus, an accelerated graphics port, and so forth. The bus(es) 718 may be associated with any suitable bus architecture including, without limitation, an Industry Standard Architecture (ISA), a Micro Channel Architecture (MCA), an Enhanced ISA (EISA), a Video Electronics Standards Association (VESA) architecture, an Accelerated Graphics Port (AGP) architecture, a Peripheral Component Interconnects (PCI) architecture, a PCI-Express architecture, a Personal Computer Memory Card International Association (PCMCIA) architecture, a Universal Serial Bus (USB) architecture, and so forth.

The memory 704 of the remote server 700 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In certain example embodiments, volatile memory may enable faster read/write access than non-volatile memory. However, in certain other example embodiments, certain types of non-volatile memory (e.g., FRAM) may enable faster read/write access than certain types of volatile memory.

In various implementations, the memory 704 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth. The memory 704 may include main memory as well as various forms of cache memory such as instruction cache(s), data cache(s), translation lookaside buffer(s) (TLBs), and so forth. Further, cache memory such as a data cache may be a multi-level cache organized as a hierarchy of one or more cache levels (L1, L2, etc.).

The data storage 720 may include removable storage and/or non-removable storage including, but not limited to, magnetic storage, optical disk storage, and/or tape storage. The data storage 720 may provide non-volatile storage of computer-executable instructions and other data. The memory 704 and the data storage 720, removable and/or non-removable, are examples of computer-readable storage media (CRSM) as that term is used herein.

The data storage 720 may store computer-executable code, instructions, or the like that may be loadable into the memory 704 and executable by the processor(s) 702 to cause the processor(s) 702 to perform or initiate various operations. The data storage 720 may additionally store data that may be copied to memory 704 for use by the processor(s) 702 during the execution of the computer-executable instructions. Moreover, output data generated as a result of execution of the computer-executable instructions by the processor(s) 702 may be stored initially in memory 704, and may ultimately be copied to data storage 720 for non-volatile storage.

More specifically, the data storage 720 may store one or more operating systems (O/S) 722; one or more database management systems (DBMS) 724; and one or more program module(s), applications, engines, computer-executable code, scripts, or the like such as, for example, one or more device tracking module(s) 726, one or more communication module(s) 728, one or more score generation module(s) 730, and/or one or more workflow management module(s) 732. Some or all of these module(s) may be sub-module(s). Any of the components depicted as being stored in data storage 720 may include any combination of software, firmware, and/or hardware. The software and/or firmware may include computer-executable code, instructions, or the like that may be loaded into the memory 704 for execution by one or more of the processor(s) 702. Any of the components depicted as being stored in data storage 720 may support functionality described in reference to correspondingly named components earlier in this disclosure.

The data storage 720 may further store various types of data utilized by components of the remote server 700. Any data stored in the data storage 720 may be loaded into the memory 704 for use by the processor(s) 702 in executing computer-executable code. In addition, any data depicted as being stored in the data storage 720 may potentially be stored in one or more datastore(s) and may be accessed via the DBMS 724 and loaded in the memory 704 for use by the processor(s) 702 in executing computer-executable code. The datastore(s) may include, but are not limited to, databases (e.g., relational, object-oriented, etc.), file systems, flat files, distributed datastores in which data is stored on more than one node of a computer network, peer-to-peer network datastores, or the like.

The processor(s) 702 may be configured to access the memory 704 and execute computer-executable instructions loaded therein. For example, the processor(s) 702 may be configured to execute computer-executable instructions of the various program module(s), applications, engines, or the like of the remote server 700 to cause or facilitate various operations to be performed in accordance with one or more embodiments of the disclosure. The processor(s) 702 may include any suitable processing unit capable of accepting data as input, processing the input data in accordance with stored computer-executable instructions, and generating output data. The processor(s) 702 may include any type of suitable processing unit including, but not limited to, a central processing unit, a microprocessor, a Reduced Instruction Set Computer (RISC) microprocessor, a Complex Instruction Set Computer (CISC) microprocessor, a microcontroller, an Application Specific Integrated Circuit (ASIC), a Field-Programmable Gate Array (FPGA), a System-on-a-Chip (SoC), a digital signal processor (DSP), and so forth. Further, the processor(s) 702 may have any suitable microarchitecture design that includes any number of constituent components such as, for example, registers, multiplexers, arithmetic logic units, cache controllers for controlling read/write operations to cache memory, branch predictors, or the like. The microarchitecture design of the processor(s) 702 may be capable of supporting any of a variety of instruction sets.

Referring now to functionality supported by the various program module(s) depicted in FIG. 7, the device tracking module(s) 726 may include computer-executable instructions, code, or the like that responsive to execution by one or more of the processor(s) 702 may perform functions including, but not limited to, tracking user interactions with documents and/or user actions at devices, generating impression pixels and/or tracking access events using user identifiers and/or device identifiers, and the like.

The communication module(s) 728 may include computer-executable instructions, code, or the like that responsive to execution by one or more of the processor(s) 702 may perform functions including, but not limited to, communicating with one or more devices, for example, via wired or wireless communication, communicating with remote servers, communicating with remote datastores, sending or receiving notifications or alerts, communicating with cache memory data, and the like.

The score generation module(s) 730 may include computer-executable instructions, code, or the like that responsive to execution by one or more of the processor(s) 702 may perform functions including, but not limited to, generating scores for user identifiers, generating scores for clinical trial site identifiers, generating scores for clinical trials, determining inspection readiness, determining graphical indications, generating user interfaces, and the like.

The workflow management module(s) 732 may include computer-executable instructions, code, or the like that responsive to execution by one or more of the processor(s) 702 may perform functions including, but not limited to, controlling access, determining permissions settings, automatically generating tasks, assigning tasks using artificial intelligence, preventing access to certain documents, and the like.

Referring now to other illustrative components depicted as being stored in the data storage 720, the O/S 722 may be loaded from the data storage 720 into the memory 704 and may provide an interface between other application software executing on the remote server 700 and hardware resources of the remote server 700. More specifically, the O/S 722 may include a set of computer-executable instructions for managing hardware resources of the remote server 700 and for providing common services to other application programs (e.g., managing memory allocation among various application programs). The O/S 722 may include any operating system now known or which may be developed in the future including, but not limited to, any server operating system, any mainframe operating system, or any other proprietary or non-proprietary operating system.

The DBMS 724 may be loaded into the memory 1004 and may support functionality for accessing, retrieving, storing, and/or manipulating data stored in the memory 704 and/or data stored in the data storage 720. The DBMS 724 may use any of a variety of database models (e.g., relational model, object model, etc.) and may support any of a variety of query languages. The DBMS 724 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like. In those example embodiments in which the remote server 700 is a mobile device, the DBMS 724 may be any suitable light-weight DBMS optimized for performance on a mobile device.

Referring now to other illustrative components of the remote server 700, the input/output (I/O) interface(s) 706 may facilitate the receipt of input information by the remote server 700 from one or more I/O devices as well as the output of information from the remote server 700 to the one or more I/O devices. The I/O devices may include any of a variety of components such as a display or display screen having a touch surface or touchscreen; an audio output device for producing sound, such as a speaker; an audio capture device, such as a microphone; an image and/or video capture device, such as a camera; a haptic unit; and so forth. Any of these components may be integrated into the remote server 700 or may be separate. The I/O devices may further include, for example, any number of peripheral devices such as data storage devices, printing devices, and so forth.

The I/O interface(s) 706 may also include an interface for an external peripheral device connection such as universal serial bus (USB), FireWire, Thunderbolt, Ethernet port or other connection protocol that may connect to one or more networks. The I/O interface(s) 706 may also include a connection to one or more of the antenna(e) 734 to connect to one or more networks via a wireless local area network (WLAN) (such as Wi-Fi) radio, Bluetooth, ZigBee, and/or a wireless network radio, such as a radio capable of communication with a wireless communication network such as a Long Term Evolution (LTE) network, WiMAX network, 3G network, ZigBee network, etc.

The remote server 700 may further include one or more network interface(s) 708 via which the remote server 700 may communicate with any of a variety of other systems, platforms, networks, devices, and so forth. The network interface(s) 708 may enable communication, for example, with one or more wireless routers, one or more host servers, one or more web servers, and the like via one or more of networks.

The antenna(e) 734 may include any suitable type of antenna depending, for example, on the communications protocols used to transmit or receive signals via the antenna(e) 734. Non-limiting examples of suitable antennas may include directional antennas, non-directional antennas, dipole antennas, folded dipole antennas, patch antennas, multiple-input multiple-output (MIMO) antennas, or the like. The antenna(e) 734 may be communicatively coupled to one or more transceivers 712 or radio components to which or from which signals may be transmitted or received.

As previously described, the antenna(e) 734 may include a cellular antenna configured to transmit or receive signals in accordance with established standards and protocols, such as Global System for Mobile Communications (GSM), 3G standards (e.g., Universal Mobile Telecommunications System (UMTS), Wideband Code Division Multiple Access (W-CDMA), CDMA2000, etc.), 4G standards (e.g., Long-Term Evolution (LTE), WiMax, etc.), direct satellite communications, or the like.

The antenna(e) 734 may additionally, or alternatively, include a Wi-Fi antenna configured to transmit or receive signals in accordance with established standards and protocols, such as the IEEE 802.11 family of standards, including via 2.4 GHz channels (e.g., 802.11b, 802.11g, 802.11n), 5 GHz channels (e.g., 802.11n, 802.11ac), or 60 GHz channels (e.g., 802.11ad). In alternative example embodiments, the antenna(e) 734 may be configured to transmit or receive radio frequency signals within any suitable frequency range forming part of the unlicensed portion of the radio spectrum.

The antenna(e) 734 may additionally, or alternatively, include a GNSS antenna configured to receive GNSS signals from three or more GNSS satellites carrying time-position information to triangulate a position therefrom. Such a GNSS antenna may be configured to receive GNSS signals from any current or planned GNSS such as, for example, the Global Positioning System (GPS), the GLONASS System, the Compass Navigation System, the Galileo System, or the Indian Regional Navigational System.

The transceiver(s) 712 may include any suitable radio component(s) for—in cooperation with the antenna(e) 734—transmitting or receiving radio frequency (RF) signals in the bandwidth and/or channels corresponding to the communications protocols utilized by the remote server 700 to communicate with other devices. The transceiver(s) 712 may include hardware, software, and/or firmware for modulating, transmitting, or receiving—potentially in cooperation with any of antenna(e) 734—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. The transceiver(s) 712 may further include hardware, firmware, or software for receiving GNSS signals. The transceiver(s) 712 may include any known receiver and baseband suitable for communicating via the communications protocols utilized by the remote server 700. The transceiver(s) 712 may further include a low noise amplifier (LNA), additional signal amplifiers, an analog-to-digital (A/D) converter, one or more buffers, a digital baseband, or the like.

The sensor(s)/sensor interface(s) 710 may include or may be capable of interfacing with any suitable type of sensing device such as, for example, inertial sensors, force sensors, thermal sensors, and so forth. Example types of inertial sensors may include accelerometers (e.g., MEMS-based accelerometers), gyroscopes, and so forth.

It should be appreciated that the program module(s), applications, computer-executable instructions, code, or the like depicted in FIG. 7 as being stored in the data storage 720 are merely illustrative and not exhaustive and that processing described as being supported by any particular module may alternatively be distributed across multiple module(s) or performed by a different module. In addition, various program module(s), script(s), plug-in(s), Application Programming Interface(s) (API(s)), or any other suitable computer-executable code hosted locally on the remote server 700, and/or hosted on other computing device(s) accessible via one or more networks, may be provided to support functionality provided by the program module(s), applications, or computer-executable code depicted in FIG. 7 and/or additional or alternate functionality. Further, functionality may be modularized differently such that processing described as being supported collectively by the collection of program module(s) depicted in FIG. 7 may be performed by a fewer or greater number of module(s), or functionality described as being supported by any particular module may be supported, at least in part, by another module. In addition, program module(s) that support the functionality described herein may form part of one or more applications executable across any number of systems or devices in accordance with any suitable computing model such as, for example, a client-server model, a peer-to-peer model, and so forth. In addition, any of the functionality described as being supported by any of the program module(s) depicted in FIG. 7 may be implemented, at least partially, in hardware and/or firmware across any number of devices.

It should further be appreciated that the remote server 700 may include alternate and/or additional hardware, software, or firmware components beyond those described or depicted without departing from the scope of the disclosure. More particularly, it should be appreciated that software, firmware, or hardware components depicted as forming part of the remote server 700 are merely illustrative and that some components may not be present or additional components may be provided in various embodiments. While various illustrative program module(s) have been depicted and described as software module(s) stored in data storage 720, it should be appreciated that functionality described as being supported by the program module(s) may be enabled by any combination of hardware, software, and/or firmware. It should further be appreciated that each of the above-mentioned module(s) may, in various embodiments, represent a logical partitioning of supported functionality. This logical partitioning is depicted for ease of explanation of the functionality and may not be representative of the structure of software, hardware, and/or firmware for implementing the functionality. Accordingly, it should be appreciated that functionality described as being provided by a particular module may, in various embodiments, be provided at least in part by one or more other module(s). Further, one or more depicted module(s) may not be present in certain embodiments, while in other embodiments, additional module(s) not depicted may be present and may support at least a portion of the described functionality and/or additional functionality. Moreover, while certain module(s) may be depicted and described as sub-module(s) of another module, in certain embodiments, such module(s) may be provided as independent module(s) or as sub-module(s) of other module(s).

Program module(s), applications, or the like disclosed herein may include one or more software components including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform.

Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component comprising instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may comprise other custom-developed application software, operating system functionality (e.g., device drivers, data storage (e.g., file management) routines, other common routines and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a computer-readable storage medium (CRSM) that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

That which is claimed is:

1. A method comprising:
determining, by one or more computer processors coupled to memory, a first score for completion of a plurality of tasks that were digitally completed by users associated with a clinical trial site identifier;
determining an average document cycle time associated with the clinical trial site identifier;
generating, based at least in part on the first score and the average document cycle time, an estimated startup time value indicative of an estimated length of time before a clinical trial can be started for the clinical trial site identifier;
determining a second score for compliance with digital tasks associated with the clinical trial site identifier;
generating a digital user interface comprising a first graphical indicator representing the first score, a second graphical indicator representing the average document cycle time, a third graphical indicator representing the estimated startup time value, and a fourth graphical indicator representing the second score; and
causing presentation of the digital user interface at a display device.

2. The method of claim 1, further comprising:
determining an inspection score for the clinical trial site identifier based at least in part on the first score and the second score;
determining that the inspection score is less than an inspection ready threshold;
presenting a graphical indicator at the digital user interface indicating that the clinical trial site identifier is not ready for inspection.

3. The method of claim 1, wherein the digital user interface further comprises graphical indicators representing inspection readiness data for a plurality of clinical trial site identifiers.

4. The method of claim 1, further comprising:
determining that a pharmaceutical monitor user account accessed the digital user interface;
storing a data record representing the access; and
automatically generating a monitoring report based at least in part on the data record.

5. The method of claim 1, further comprising:
receiving a request to submit clinical trial data;
determining that an error is present in the clinical trial data;
preventing submission of the clinical trial data; and
generating a series of guided user interfaces to correct the error.

6. The method of claim 1, further comprising:
determining an average time to completion for the plurality of tasks;
determining a number of uncompleted tasks associated with the clinical trial site identifier; and
determining a number of tasks completed after respective deadlines;
wherein determining the first score comprises determining the first score based at least in part on the average time, the number of uncompleted tasks, and the number of tasks completed after the respective deadlines.

7. The method of claim 1, further comprising:
determining that a first task associated with a first document is assigned to a first user identifier;
receiving an indication that the first user identifier has requested access to the first document from a user device;
receiving an indication from the user device that a digital action was completed on the first document;
determining that the first task is completed;
modifying a state associated with the first task to indicate that the first task is complete;
determining a second task based at least in part on the state; and
assigning the second task to a second user identifier.

8. The method of claim 7, further comprising:
determining a length of time between a time of assignment of the first task to the first user and a time of completion of the first task;
determining a number of late tasks associated with the first user identifier;
determining a number of completed tasks associated with the first user identifier; and
generating a performance score for the first user identifier based at least in part on the length of time, the number of late tasks, and the number of completed tasks;
wherein a level of access associated with the first user identifier is based at least in part on the performance score.

9. The method of claim 7, further comprising:
determining that a deadline to complete the first task has elapsed;
receiving an indication that the first user identifier has requested access to a second document; and
preventing access to the second document by the first user identifier until the first task is completed.

10. A device comprising:
memory that stores computer-executable instructions; and
at least one processor configured to access the memory and execute the computer-executable instructions to:
determine a first score for completion of a plurality of tasks that were digitally completed by users associated with a clinical trial site identifier;
determine an average document cycle time associated with the clinical trial site identifier;
generate, based at least in part on the first score and the average document cycle time, an estimated startup time value indicative of an estimated length of time before a clinical trial can be started for the clinical trial site identifier;
determine a second score for compliance with digital tasks associated with the clinical trial site identifier;
generate a digital user interface comprising a first graphical indicator representing the first score, a second graphical indicator representing the average document cycle time, a third graphical indicator representing the estimated startup time value, and a fourth graphical indicator representing the second score; and
present the digital user interface at a display device.

11. The device of claim 10, wherein the at least one processor is further configured to access the memory and execute the computer-executable instructions to:
determine an inspection score for the clinical trial site identifier based at least in part on the first score and the second score;
determine that the inspection score is less than an inspection ready threshold;
present a graphical indicator at the digital user interface indicating that the clinical trial site identifier is not ready for inspection.

12. The device of claim 10, wherein the digital user interface further comprises graphical indicators representing inspection readiness data for a plurality of clinical trial site identifiers.

13. The device of claim 10, wherein the at least one processor is further configured to access the memory and execute the computer-executable instructions to:
determine that a pharmaceutical monitor user account accessed the digital user interface;
store a data record representing the access; and
automatically generate a monitoring report based at least in part on the data record.

14. The device of claim 10, wherein the at least one processor is further configured to access the memory and execute the computer-executable instructions to:
receive a request to submit clinical trial data;
determine that an error is present in the clinical trial data;
prevent submission of the clinical trial data; and
generate a series of guided user interfaces to correct the error.

15. The device of claim 10, wherein the at least one processor is further configured to access the memory and execute the computer-executable instructions to:
determine an average time to completion for the plurality of tasks;
determine a number of uncompleted tasks associated with the clinical trial site identifier; and
determine a number of tasks completed after respective deadlines;
wherein the at least one processor is configured to determine the first score by executing the computer-executable instructions to determine the first score based at least in part on the average time, the number of uncompleted tasks, and the number of tasks completed after the respective deadlines.

16. The device of claim 10, wherein the at least one processor is further configured to access the memory and execute the computer-executable instructions to:
determine that a first task associated with a first document is assigned to a first user identifier;
receive an indication that the first user identifier has requested access to the first document from a user device;
receive an indication from the user device that a digital action was completed on the first document;
determine that the first task is completed;
modify a state associated with the first task to indicate that the first task is complete;
determine a second task based at least in part on the state; and
assign the second task to a second user identifier.

17. The device of claim 16, wherein the at least one processor is further configured to access the memory and execute the computer-executable instructions to:
determine a length of time between a time of assignment of the first task to the first user and a time of completion of the first task;
determine a number of late tasks associated with the first user identifier;
determine a number of completed tasks associated with the first user identifier; and
generate a performance score for the first user identifier based at least in part on the length of time, the number of late tasks, and the number of completed tasks;
wherein a level of access associated with the first user identifier is based at least in part on the performance score.

18. The device of claim 10, wherein the at least one processor is further configured to access the memory and execute the computer-executable instructions to:
determine that a deadline to complete the first task has elapsed;
receive an indication that the first user identifier has requested access to a second document; and
prevent access to the second document by the first user identifier until the first task is completed.

19. A method comprising:
determining, by one or more computer processors coupled to memory, a first score for completion of a plurality of tasks that were digitally completed by users associated with a clinical trial site identifier;
determining an average document cycle time associated with the clinical trial site identifier;
determining a second score for compliance with digital tasks associated with the clinical trial site identifier;
generating a digital user interface comprising a first graphical indicator representing the first score, a second graphical indicator representing the estimated startup time value, and a third graphical indicator representing the second score; and
causing presentation of the digital user interface at a display device.

20. The method of claim 19, further comprising:
determining a clinical trial identifier associated with the clinical trial site identifier; and
generating a third score for the clinical trial identifier, wherein the third score at least partially represents completion of milestones associated with the clinical trial identifier.
